(19) United States
(12) Patent Application Publication
(10) Pub. No.: US 2026/0050053 A1
(43) Pub. Date: Feb. 19, 2026

(54) METHOD FOR SIMULTANEOUS IMAGING AND IMAGE PROCESSING OF NEUROMELANIN AND NIGROSOME 1 USING 3D MULTI-ECHO GRE

(71) Applicant: Heuron Co., Ltd., Seoul (KR)

(72) Inventors: Sungmin GHO, Yongin-si (KR); Hwan HEO, Seongnam-si (KR); Aleum LEE, Seoul (KR); Jeongwon JO, Incheon (KR)

(21) Appl. No.: 18/896,104

(22) Filed: Sep. 25, 2024

(30) Foreign Application Priority Data
Aug. 16, 2024 (KR) 10-2024-0109566

(51) Int. Cl. G01R 33/56; A61B 5/00; A61B 5/055; G06T 7/00; G06T 7/11; G06T 7/149

(52) U.S. Cl. CPC G01R 33/5608; A61B 5/0042; A61B 5/055; A61B 5/4064; A61B 5/4082; G06T 7/0012; G06T 7/11; G06T 7/149; G06T 2207/10088; G06T 2207/20084; G06T 2207/30016

(57) ABSTRACT

A method for simultaneous imaging and image processing of neuromelanin and nigrosome 1 using 3D multi-echo GRE performed by a computer includes: a step (S120) of acquiring a neuromelanin (NM) image (130) and a multi-echo gradient recalled echo (GRE) brain image with a more enhanced contrast ratio than an MRI image by applying two spatial saturation pulses to a 3D multi-echo GRE; a step (S150) of generating a susceptibility map weighted imaging (SMWI) image (140) for nigrosome 1 (N1) using the 3D multi-echo GRE image acquired in the acquisition step (S120); and an inference step (S170) of analyzing, by a neural network (NN) model, the NM image (130) and the SMWI image (140) to quantify volumes of the neuromelanin (NM) and the nigrosome 1 (N1).

### CROSS-REFERENCE TO RELATED APPLICATIONS

This application claims the priority of Korean Patent Application No. 10-2024-0109566 filed on Aug. 16, 2024, in the Korean Intellectual Property Office, the disclosure of which is incorporated herein by reference.

### BACKGROUND

### Field

The present disclosure relates to a simultaneous imaging technique of a neuromelanin area and a nigrosome 1 area, and more particularly, to a method for simultaneous imaging and image processing of neuromelanin and nigrosome 1 using 3D multi-echo gradient recalled echo (GRE).

### Description of the Related Art

In general, the Parkinson's disease is one of the common neurodegenerative diseases, and occurs due to degenerative degeneration and loss of dopaminergic neurons (a type of catecholaminergic neurons) located in a substantia nigra (SN) of a midbrain which plays a role in controlling movement For this reason, tracking the degeneration of the dopaminergic neurons is an essential element in diagnosing the Parkinson's disease and tracking stages of the Parkinson's disease.

In addition, the neuromelanin (NM) is a protein polymer synthesized to protect the dopaminergic neurons from oxidative stress. In a dopamine synthesis process of the dopaminergic neurons, the dopamine remaining in cytoplasm of the dopaminergic neurons undergoes autooxidation using iron as a catalyst or copper as an indirect catalyst. This autooxidation had the problem of causing damage by causing the oxidative stress to organelles. To protect cells from the stress, the neuromelanin is synthesized in the cytoplasm to capture iron. In this case, an NM-iron complex is formed. The degeneration of the dopaminergic neurons may be confirmed by the degree of deposition of the neuromelanin due to the complex. Due to these characteristics, the neuromelanin is used as an important biomarker for tracking the degeneration of the dopaminergic neurons.

However, among the conventional MRI images, a neuromelanin-MRI (NM-MRI) image obtained by imaging the substantia nigra area has low contrast, so accurate de-deposition information on the neuromelanin may not be obtained.

In addition, nigrosome 1 (N1) is a medical term referring to a very small area in substantia nigra pars compacta within the substantia nigra (SN). The substantia nigra pars compacta are a midbrain structure that includes a high-density population of the dopaminergic neurons. These dopaminergic neurons are progressively lost in idiopathic Parkinson's disease (IPD), resulting in disability. This area shows increased iron concentration in IPD patients when compared with a healthy control. The contrast between the nigrosome 1 and the surrounding substantia nigra area is due to the difference in iron concentration, and the difference in magnetic susceptibility between two areas is known to be significantly reduced in the IPD patients. Therefore, the reduced difference in magnetic susceptibility between the nigrosome 1 and the surrounding substantia nigra area has been used as an imaging biomarker in the IPD patients. Therefore, the nigrosome 1 structures have been successfully depicted on 7T (Tesla) MRI using high-resolution (e.g., 0.3 mm in-plane resolution) T2-weighted image or susceptibility-weighted imaging (SWI). However, there was a problem in that the contrast was significantly reduced in the 3D high-resolution T2-weighted image due to the limited spatial resolution and signal/contrast-to-noise ratio (SNR/CNR) in the low magnetic field strength such as 3T MRI. Despite several successful studies that demonstrated the usefulness of the approach, these limitations often hindered the reliability and applicability of the nigrosome 1 imaging in the 3T MRI.

As such, the need for the imaging of the neuromelanin and nigrosome 1 was greatly raised, but each imaging had a different contrast mechanism, and therefore, should be not performed simultaneously, but performed separately. That is, there is a mechanism in which the contrast of the neuromelanin is enhanced using a magnetization transfer (MT) pulse, and the contrast of the nigrosome 1 is enhanced through sensitivity weights to iron distributed therearound.

Due to these technical differences, in order to image the neuromelanin and nigrosome 1, each should be scanned (imaged) sequentially, which has the disadvantage that the scan time takes 10 minutes or more.

### RELATED ART DOCUMENT

### Patent Document

1. US Patent Laid-Open Publication No. 2022-0273183 (neuromelanin-sensitive MRI for evaluating Parkinson's disease),
2. U.S. Pat. No. 8,781,197 (Tool for accurate quantification in molecular MRI)

### SUMMARY

An object to be achieved by the present disclosure is to provide a method for simultaneous imaging and image processing of neuromelanin and nigrosome 1 using 3D multi-echo gradient recalled echo (GRE) capable of simultaneously imaging the neuromelanin and nigrosome 1 which are helpful in diagnosing the Parkinson's disease.

Another object to be achieved by the present disclosure is to provide a method for simultaneous imaging and image processing of neuromelanin and nigrosome 1 using 3D multi-echo GRE capable of accurately parceling and quantifying an area of interest by using a neural network model based on deep learning for quantification of the neuromelanin and nigrosome 1 in the image processing.

However, objects of the present disclosure are not limited to the above-described objects. That is, other objects that are not described may be obviously understood by those skilled in the art to which the present disclosure pertains from the following description.

According to an aspect of the present disclosure, there is provided a method for simultaneous imaging and image processing of neuromelanin and nigrosome 1 using 3D multi-echo GRE performed by a computer, including: a step (S120) of acquiring a neuromelanin (NM) image 130 and a multi-echo gradient recalled echo (GRE) brain image with a more enhanced contrast ratio than an MRI image by applying two spatial saturation pulses to a 3D multi-echo GRE; a step (S150) of generating a susceptibility map weighted imaging (SMWI) image 140 for nigrosome 1 (N1) using the 3D multi-echo GRE image acquired in the acquisition step (S120); and an inference step (S170) of analyzing, by a neural network (NN) model, the NM image 130 and the SMWI image 140 to quantify volumes of the neuromelanin (NM) and the nigrosome 1 (N1).

Further, in the acquisition step (S120), a first echo image of the 3D multi-echo GRE may be a neuromelanin (NM) image with an enhanced contrast ratio.

Further, in the acquisition step (S120), in order to enhance the contrast ratio of the nigrosome 1 (N1), the SMWI image may be generated by applying a sensitivity weight to an area containing iron (Fe) to second and subsequent echo data of the 3D multi-echo GRE.

Further, the sensitivity weight for the area containing iron (Fe) may be applied to second to fourth echo data of the 3D multi-echo GRE.

Further, the neural network (NN) model may be constructed by a step (S200) of constructing a model based on a convolutional neural network (convolutional NN) and a template and quantifying the neuromelanin (NM) and the nigrosome 1 (N1); a step (S220) of generating a template of the neuromelanin (NM) and the nigrosome 1 (N1); a step (S230) of modifying an inference model of the nigrosome 1 (N1) using quantitative volume data of the nigrosome 1 (N1) and the convolutional neural network (CNN) model and a step (S240) of constructing a segmentation model for substantia nigra segmentation and a parcellation model for spatial normalization of that neuromelanin (NM) image that are performed in parallel; a step (S250) of training the N1 inference model, the segmentation model, and the parcellation model using the neuromelanin (NM) image, the SMWI image, and the healthy control (HC) data; and a step (S260) of completing the N1 inference model, the segmentation model, and the parcellation model.

Further, the neuromelanin (NM) image and the SMWI image may include data for an idiopathic Parkinson's disease (IPD).

Further, the parcellation model may parcel a brain of the patient into midbrain, pons, brainstem, substantia nigra (SN), and periaqueductal gray (PAG).

Further, the acquisition step (S120) may be executed within 5 minutes.

Further, in the inference step (S170), the neural network (NN) model may detect a substantia nigra area from the NM image 130 to numerically quantify and infer a degree of de-deposition of neuromelanin (NM).

According to an embodiment of the present disclosure, it is possible to simultaneously image the neuromelanin and nigrosome 1 which are helpful in diagnosing the Parkinson's disease. Therefore, there is an advantage of shortening the conventional scan time, which took 10 minutes or more, to less than 5 minutes.

In addition, by using the deep learning-based neural network model to quantify the neuromelanin and nigrosome 1, it is possible to accurately parcel and quantify the area of interest. This allows the medical staff to diagnose the Parkinson's disease more accurately.

In addition, since a large amount of image data may be trained through the artificial intelligence and the deep learning, and the model may always be maintained as the latest model, it is possible to escape from the conventional method that relied on the knowledge and experience of experts.

In addition, the images quantified and provided according to the present disclosure enable visualization of clear signals (e.g., swallowtail signal loss, magnetization transfer-driven change in the substantia nigra). As a result, it is possible to diagnose the Parkinson's disease earlier than conventional determination method.

However, effects which can be achieved by the present disclosure are not limited to the above-described effects. That is, other objects that are not described may be obviously understood by those skilled in the art to which the present disclosure pertains from the following description.

### BRIEF DESCRIPTION OF THE DRAWINGS

The above and other aspects, features and other advantages of the present disclosure will be more clearly understood from the following detailed description taken in conjunction with the accompanying drawings, in which:

FIG. 1 is a flowchart of a method for simultaneous imaging and image processing of neuromelanin and nigrosome 1 using 3D multi-echo gradient recalled echo (GRE) according to an embodiment of the present disclosure;

FIG. 2 is a flowchart schematically illustrating a process from construction to completion of a neural network model according to an embodiment of the present disclosure;

FIG. 3 is a conceptual diagram schematically illustrating an imaging plan according to the present disclosure;

FIG. 4 is a conceptual diagram illustrating a specific operation concept of an imaging slab in FIG. 3;

FIG. 5 is an example of a neuromelanin (NM) template used in the present disclosure;

FIG. 6A is an example of a neuromelanin (NM) image with an enhanced contrast ratio for a healthy control (HC) according to the present disclosure;

FIG. 6B is an example of the neuromelanin (NM) image with an enhanced contrast ratio for an idiopathic Parkinson's disease (IPD) patient according to the present disclosure;

FIG. 7A is a graph comparing neuromelanin (NM) volumes in the healthy control group (Non PD) and the Parkinson's disease (PD) patient;

FIG. 7B is a graph comparing the contrast ratios of the neuromelanin (NM) images in the healthy control group (Non PD) and the Parkinson's disease (PD) patient;

FIG. 8A is an example of a nigrosome 1 (N1) image with an enhanced contrast ratio for the healthy control (HC) according to the present disclosure;

FIG. 8B is an example of the nigrosome 1 (N1) image with an enhanced contrast ratio for the idiopathic Parkinson's disease (IPD) patient according to the present disclosure;

FIGS. 9A, 10A, and FIG. 11A are examples of images processed by a parcellation model of the present disclosure; and

FIGS. 9B, 10B, and 11B are examples of images in different planes for FIGS. 9A, 10A, and 11A, respectively.

### DETAILED DESCRIPTION OF THE PREFERRED EMBODIMENT

Hereinafter, embodiments of the present disclosure will be described in detail with reference to the accompanying drawings so that those skilled in the art to which the present disclosure pertains may easily practice the present disclosure. However, description of the present disclosure is only an embodiment for structural or functional description, and therefore the scope of the present disclosure should not be construed as limited to embodiments described in the text. That is, since the embodiments may be variously modified and may have various forms, the scope of the present disclosure should be construed as including equivalents capable of realizing the technical idea. In addition, a specific embodiment is not construed as including all the objects or effects presented in the present disclosure or only the effects, and therefore the scope of the present disclosure should not be understood as being limited thereto.

The meanings of terms described in the present disclosure should be understood as follows.

Terms such as “first” and “second” are intended to distinguish one component from another component, and the scope of the present disclosure should not be limited by these terms. For example, a first component may be named a second component and the second component may also be similarly named the first component. It is to be understood that when one element is referred to as being “connected to” another element, it may be connected directly to or coupled directly to another element or be connected to another element, having the other element intervening therebetween. On the other hand, it is to be understood that when one element is referred to as being “connected directly to” another element, it may be connected to or coupled to another element without the other element intervening therebetween. Meanwhile, other expressions describing a relationship between components, that is, “between”, “directly between”, “neighboring to”, “directly neighboring to” and the like, should be similarly interpreted.

It should be understood that the singular expression include the plural expression unless the context clearly indicates otherwise, and it will be further understood that the terms “comprises” or “have” used in this specification, specify the presence of stated features, steps, operations, components, parts, or a combination thereof, but do not preclude the presence or addition of one or more other features, numerals, steps, operations, components, parts, or a combination thereof.

Unless defined otherwise, all the terms used herein including technical and scientific terms have the same meaning as meanings generally understood by those skilled in the art to which the present disclosure pertains. It should be understood that the terms defined by the dictionary are identical with the meanings within the context of the related art, and they should not be ideally or excessively formally defined unless the context clearly dictates otherwise.

### Configuration of Embodiment

Hereinafter, configurations of an exemplary embodiment of the present disclosure will be described in detail with reference to the accompanying drawings. An image processing method of the present disclosure may be implemented by a computer and software.

The software may be an image processing program, an operating system (e.g., a Windows program), a neural network learning program, etc.

A control unit of the computer controls the above-described image processing program, operating system (e.g., a Windows program), and neural network learning program, and may be a microcomputer, a CPU, an application processor (AP), etc. In addition, the computer includes a data storage device (hard disk, flash memory, RAM, ROM, etc.), an input device (keyboard, mouse, touch screen, USB port, etc.), an output device (display monitor, speaker), a communication device (Ethernet port, LAN port, wireless communication port, etc.).

### Operation of Embodiment

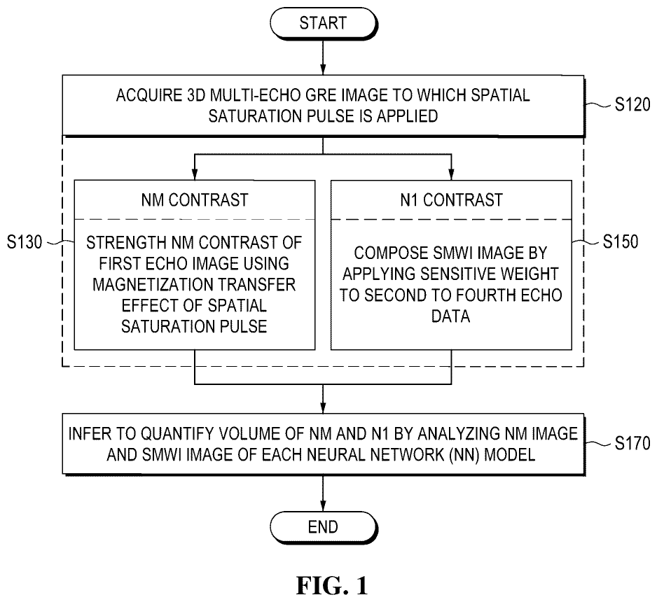

Hereinafter, operations of an exemplary embodiment of the present disclosure will be described in detail with reference to the accompanying drawings. FIG. 1 is a flowchart of a method for simultaneous imaging and image processing of neuromelanin and nigrosome 1 using 3D multi-echo GRE according to an embodiment of the present disclosure. As illustrated in FIG. 1, a neuromelanin (NM) image 130 and a multi-echo gradient recalled echo (GRE) image with a more enhanced contrast ratio than a general MRI image are acquired by applying two spatial saturation pulses to a 3D multi-echo GRE (S120). The acquisition step (S120) may be executed within 5 minutes. In this case, any MRI equipment with low magnetic field strength of 3T (Tesla) or less may be used. The imaged MRI image is transmitted to the computer and stored.

Next, a susceptibility map weighted imaging (SMWI) image 140 for nigrosome 1 (N1) is generated using the 3D multi-echo GRE image acquired through the above process (S150).

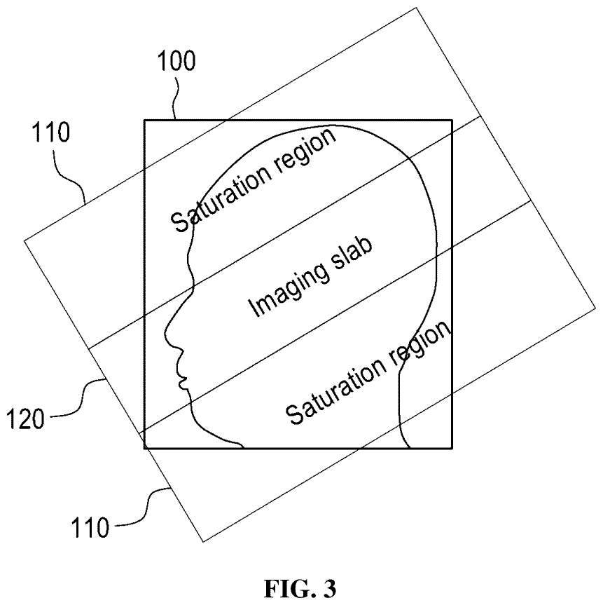

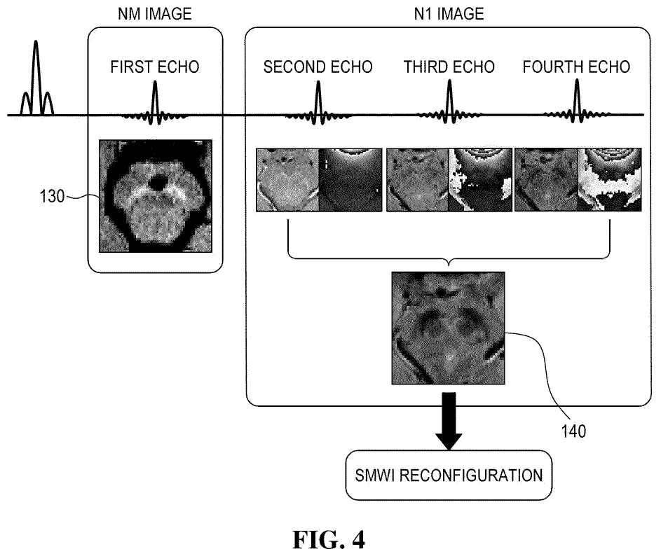

FIG. 3 is a conceptual diagram schematically illustrating an imaging plan according to the present disclosure, and FIG. 4 is a conceptual diagram illustrating a specific operation concept of the 3D multi-echo GRE of an imaging slab in FIG. 3. As illustrated in FIGS. 3 and 4, a plurality of echo signals (first echo, second echo, third echo, fourth echo, etc.) are output due to a 3D multi-echo GRE sequence.

Then, in the acquisition step (S120), a first echo magnitude image of the 3D multi-echo GRE is the neuromelanin (NM) image 130 (S130).

Further, the processing for the N1 image is as follows. In order to enhance a contrast ratio of nigrosome 1 (N1), a sensitivity weight based on quantitative susceptibility mapping) for an area containing iron (Fe) is applied to second to fourth echo magnitude and phase data of the 3D multi-echo GRE to generate a susceptibility map weighted imaging (SMWI) image 140 (S150). That is, a total of six images are combined to construct one SMWI image 140: two images derived from the second echo data, two images derived from the third echo data, and two images derived from the fourth echo data.

Then, a neural network (NN) model analyzes the NM image 130 and the SMWI image 140 with an enhanced contrast ratio to infer quantification of volumes of the neuromelanin (NM) and the nigrosome 1 (N1) (S170). That is, the neural network (NN) model may detect a substantia nigra area from the NM image 130 to numerically quantify and infer a degree of de-deposition of the neuromelanin (NM).

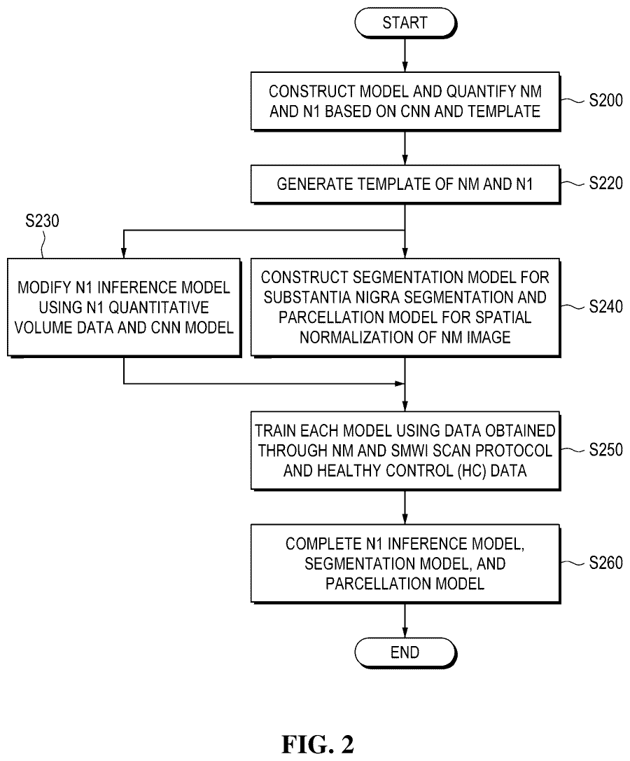

This neural network is completed through the following process. FIG. 2 is a flowchart schematically illustrating a process from construction to completion of a neural network model according to an embodiment of the present disclosure. As illustrated in FIG. 2, first, a model is constructed based on a convolutional neural network (convolutional NN) and a template and the neuromelanin (NM) and the nigrosome 1 (N1) are quantified (S200).

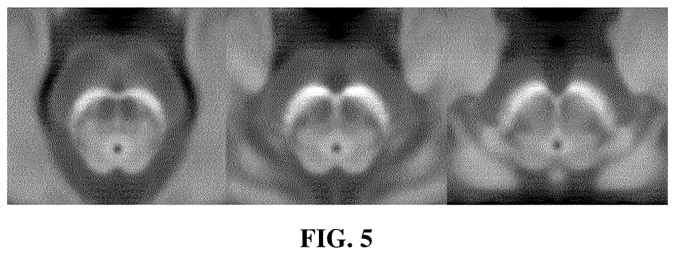

Next, the templates of the neuromelanin (NM) and the nigrosome 1 (N1) are each generated (S220). FIG. 5 is an example of the neuromelanin (NM) template used in the present disclosure.

Next, the quantitative volume data of the nigrosome 1 (N1) and the convolutional neural network (CNN) model are used to modify an inference model of the nigrosome 1 (N1) (S230). Separately, a segmentation model for substantia nigra segmentation and a parcellation model for spatial normalization of the neuromelanin (NM) image are constructed (S240).

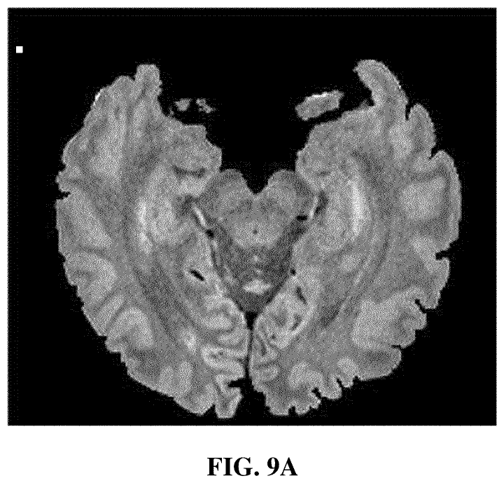

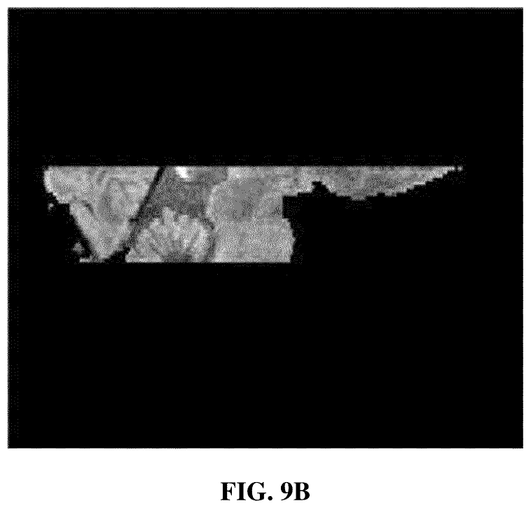

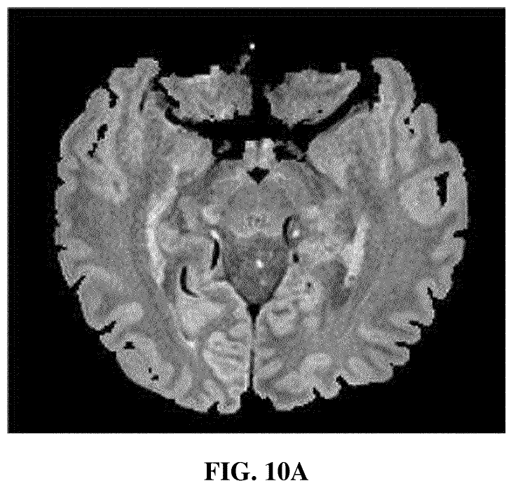

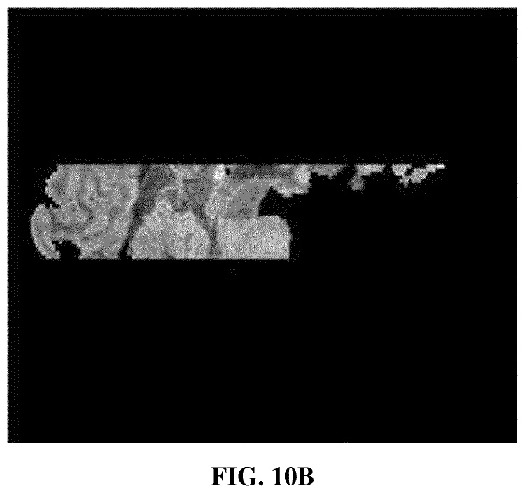

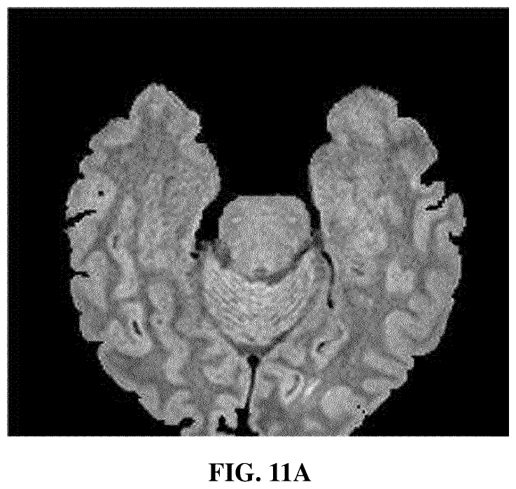

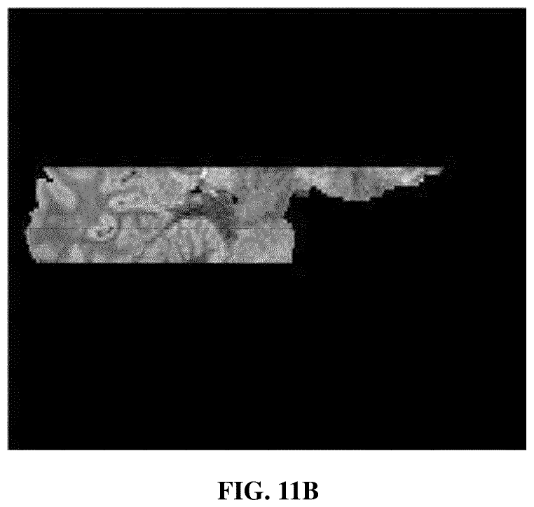

In particular, the parcellation model may parcel a brain of the patient into midbrain, pons, brainstem, substantia nigra (SN), and periaqueductal gray (PAG). FIGS. 9A, 10A, and 11a are examples of images processed by the parcellation model of the present disclosure, and FIGS. 9B, 10B, and 11B are examples of images in different planes for FIGS. 9A, 10A, and 11A.

These steps S230 and S240 may be performed simultaneously in parallel or sequentially or in reverse order.

Then, the N1 inference model, the segmentation model, and the parcellation model are each trained using the neuromelanin (NM) image, the SMWI image, and the healthy control (HC) data (S250). In the training process, the neuromelanin (NM) image and the SMWI image may include data for an idiopathic Parkinson's disease (IPD) and/or a Parkinson's disease.

When the training process through the learning converges into a certain range, the N1 inference model, the segmentation model, and the parcellation model are completed (S260).

That is, the completed segmentation model segments the substantia nigra in the MRI image, and the parcellation model normalizes a degree of de-deposition of the neuromelanin (NM) in the NM image 130 and outputs the degree of de-deposition as a number or graphic. Then, the N1 inference model normalizes a difference in magnetic susceptibility from the SMWI image 140 and outputs the normalized difference in magnetic susceptibility as a number or graphic.

### Experimental Embodiment

Hereinafter, the results of specific experimental embodiments according to the present disclosure will be described. First, experimental conditions of a 3D multi-echo GRE sequence used in the experiment of the present disclosure are as follows.
Spatial saturation pulses are respectively placed above and below the image volume.
Thickness=80 mm, gap=10 mm, temporal resolution=60 ms, the number of echoes=4, echo time=4, 14, 26.5, 38.5 ms, illuminating angle=20°, voxel size=0.8×0.8×1 mm3, the number of slices=32, acceleration factor=2, scan (imaging) time=4 minutes 21 seconds.

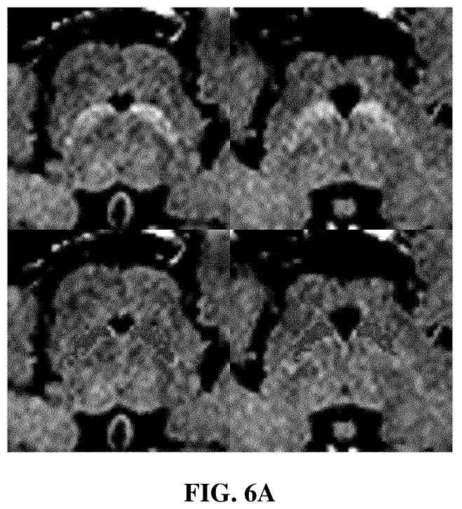

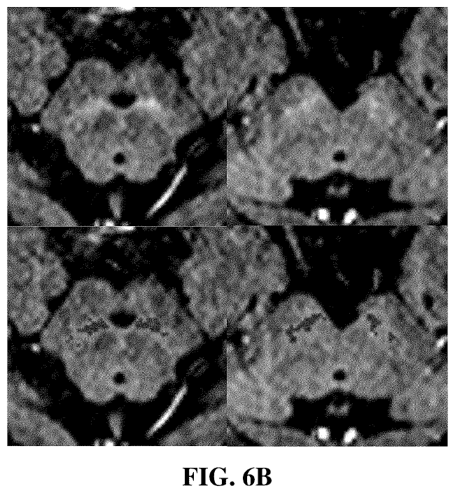

FIG. 6A is an example of a neuromelanin (NM) image with an enhanced contrast ratio for a healthy control (HC) according to the present disclosure, and FIG. 6B is an example of the neuromelanin (NM) image with an enhanced contrast ratio for an idiopathic Parkinson's disease (IPD) patient according to the present disclosure. In FIGS. 6A and 6B, green represents a high-intensity area segmented by the inference of the neural network model of the present disclosure. It could be confirmed through FIGS. 6A and 6B that the neural network model of the present disclosure accurately segments the substantia nigra (SN) area.

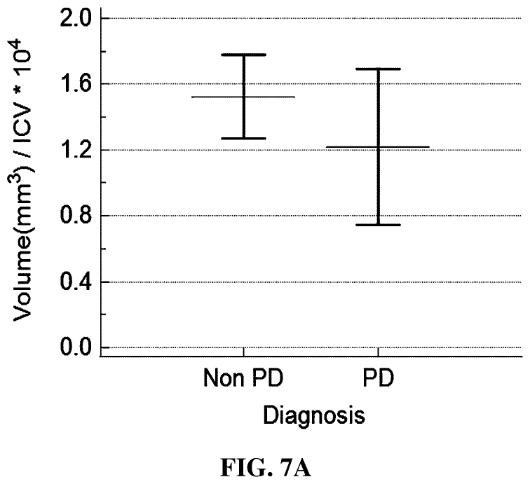

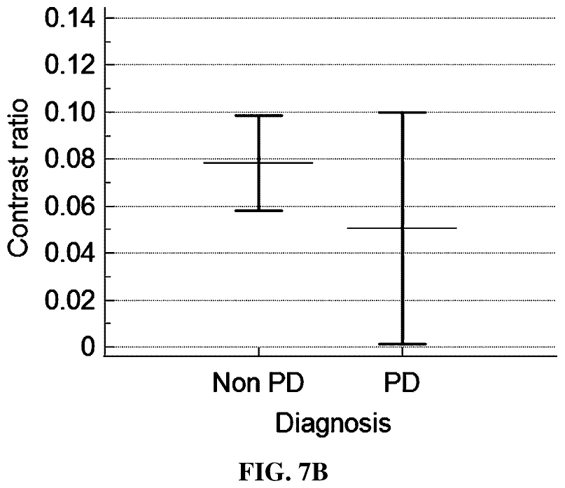

FIG. 7A is a graph comparing neuromelanin (NM) volumes in the healthy control group (Non PD) and the Parkinson's disease (PD) patient, and FIG. 7B is a graph comparing the contrast ratios of the neuromelanin (NM) images in the healthy control group (Non PD) and the Parkinson's disease (PD) patient. A vertical axis of FIG. 7A is a value obtained by dividing the NM volume by intracranial volume (ICV)×10,000. The ICV is calculated by measuring a size of a brain excluding a cerebrospinal fluid (CSF) from a T1-weighted image.

As may be seen from FIG. 7A, the healthy control group (Non PD) had a value of 1.53±0.27, and the Parkinson's disease (PD) patient had a value of 1.22±0.48. This indicates that the Parkinson's disease (PD) patient has a volume reduction of 20% compared to the healthy control group (Non PD).

As may be seen from FIG. 7B, the healthy control group (Non PD) had a contrast ratio of 0.08±0.02, and the Parkinson's disease (PD) patient had a contrast ratio of 0.05±0.04. This indicates that the Parkinson's disease (PD) patient has a contrast ratio reduction of 35% compared to the healthy control group (Non PD).

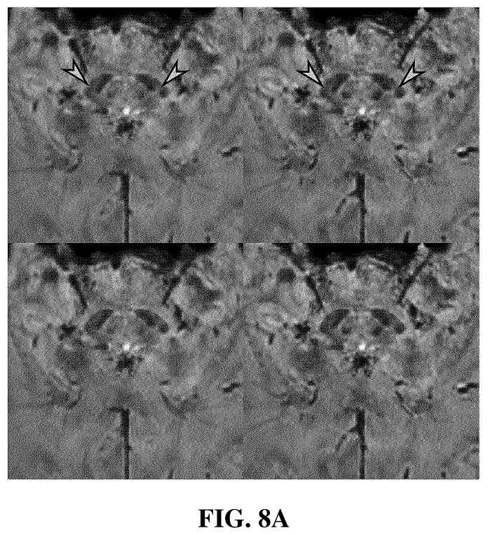

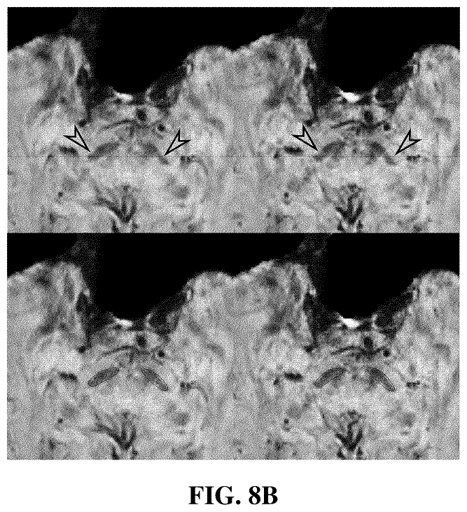

FIG. 8A is an example of a nigrosome 1 (N1) image with an enhanced contrast ratio for the healthy control (HC) according to the present disclosure, and FIG. 8B is an example of the nigrosome 1 (N1) image with an enhanced contrast ratio for the idiopathic Parkinson's disease (IPD) patient according to the present disclosure. In FIGS. 8A and 8B, a blue closed curve indicated by a yellow arrow represents an area automatically segmented in the substantia nigra (SN) by the segmentation model of the present disclosure, and a red closed curve represents an area automatically segmented in the N1 image by the segmentation model of the present disclosure.

As illustrated in FIGS. 8A and 8B, a clear swallowtail shape was observed in the healthy control group (HC) as in FIG. 8A. However, in the idiopathic Parkinson's disease (IPD) patients as in FIG. 8B, the swallowtail shape disappeared on both sides. In addition, it may be observed that the N1 area of the idiopathic Parkinson's disease (IPD) patients decreases.

In addition, [Table 1] shows the diagnostic performance of the neural network model according to the present disclosure for N1 classification for all participants. (GT: 18F-FP-CIT PET)

TABLE 1

| Sensitivity, | Specificity, | | | Accuracy, |
|---|---|---|---|---|
| % | % | PPV, % | NPV, % | % |
| 90.9 | 94.4 | 90.9 | 94.4 | 93.1 |
| [58.7, 99.8] | [72.7, 99.9] | [59.6, 98.5] | [72.3, 99.1] | [77.2, 99.2] |

Here, Values represent mean [range]
PPV: Positive predictive value
NPV: Negative predictive value

As shown in [Table 1], all estimates for sensitivity, specificity, a positive predictive value (PPV), a negative predictive value (NPV), and accuracy are 90% or more. This indicates that the neural network model according to the present disclosure is correctly constructed and accurately infers.

A detailed description of preferred embodiments of the invention disclosed as described above is provided to enable a person skilled in the art to implement or practice the invention. Although exemplary embodiments of the present disclosure have been disclosed above, it may be understood by those skilled in the art that the present disclosure may be variously modified and changed without departing from the scope of the present disclosure. For example, a person skilled in the art may use each configuration described in the above-described embodiments by combining them with each other. Accordingly, the present disclosure is not intended to be limited to the embodiments illustrated herein but is to be given the widest scope consistent with the principles and novel features disclosed herein.

The present disclosure may be implemented in another specific form without departing from the spirit and the essential feature of the present disclosure. Therefore, the above-mentioned detailed description is to be interpreted as being illustrative rather than being restrictive in all aspects. The scope of the present disclosure is to be determined by reasonable interpretation of the claims, and all modifications within an equivalent range of the present disclosure fall in the scope of the present disclosure. The present disclosure is not intended to be limited to the embodiments illustrated herein but is to be given the widest scope consistent with the principles and novel features disclosed herein. In addition, claims that do not have an explicit reference relationship in the patent claims may be combined to form an embodiment or may be included as a new claim through amendment after filing.

What is claimed is:

1. A method for simultaneous imaging and image processing of neuromelanin and nigrosome 1 using 3D multi-echo GRE performed by a computer, the method comprising:
a step (S120) of acquiring a neuromelanin (NM) image (130) and a multi-echo gradient recalled echo (GRE) brain image with a more enhanced contrast ratio than an MRI image by applying two spatial saturation pulses to a 3D multi-echo GRE;
a step (S150) of generating a susceptibility map weighted imaging (SMWI) image (140) for nigrosome 1 (N1) using the 3D multi-echo GRE image acquired in the acquisition step (S120); and
an inference step (S170) of analyzing, by a neural network (NN) model, the NM image (130) and the SMWI image (140) to quantify volumes of the neuromelanin (NM) and the nigrosome 1 (N1).

2. The method according to claim 1, wherein in the acquisition step (S120), a first echo image of the 3D multi-echo GRE is a neuromelanin (NM) image with an enhanced contrast ratio.

3. The method according to claim 1, wherein in the acquisition step (S120), in order to enhance the contrast ratio of the nigrosome 1 (N1), the SMWI image is generated by applying a sensitivity weight to an area containing iron (Fe) to second and subsequent echo data of the 3D multi-echo GRE.

4. The method according to claim 3, wherein the sensitivity weight for the area containing iron (Fe) is applied to second to fourth echo data of the 3D multi-echo GRE.

5. The method according to claim 1, wherein the neural network (NN) model is constructed by a step (S200) of constructing a model based on a convolutional neural network (convolutional NN) and a template and quantifying the neuromelanin (NM) and the nigrosome 1 (N1);
a step (S220) of generating a template of the neuromelanin (NM) and the nigrosome 1 (N1);
a step (S230) of modifying an inference model of the nigrosome 1 (N1) using quantitative volume data of the nigrosome 1 (N1) and the convolutional neural network (CNN) model and a step (S240) of constructing a segmentation model for substantia nigra segmentation and a parcellation model for spatial normalization of that neuromelanin (NM) image that are performed in parallel;
a step (S250) of training the N1 inference model, the segmentation model, and the parcellation model using the neuromelanin (NM) image, the SMWI image, and the healthy control (HC) data; and
a step (S260) of completing the N1 inference model, the segmentation model, and the parcellation model.

6. The method according to claim 5, wherein the neuromelanin (NM) image and the SMWI image include data for an idiopathic Parkinson's disease (IPD).

7. The method according to claim 5, wherein the parcellation model parcels a brain of the patient into midbrain, pons, brainstem, substantia nigra (SN), and periaqueductal gray (PAG).

8. The method according to claim 1, wherein the acquisition step (S120) is executed within 5 minutes.

9. The method according to claim 1, wherein in the inference step (S170), the neural network (NN) model detects a substantia nigra area from the NM image (130) to numerically quantify and infer a degree of de-deposition of the neuromelanin (NM).
